(19) United States
(12) Patent Application Publication
(10) Pub. No.: US 2026/0048192 A1
(43) Pub. Date: Feb. 19, 2026

(54) FLEXIBLE CONNECTOR

(71) Applicant: MEDICUD S.R.L., Rome (RM) (IT)

(72) Inventors: Andrea CAZZULANI, Rome (RM) (IT); Antonio D'ANGELO, Rome (RM) (IT); Cristina SCARANO, Rome (RM) (IT)

(73) Assignee: MEDICUD S.R.L., Rome (RM) (IT)

(21) Appl. No.: 19/105,230

(22) PCT Filed: Sep. 11, 2023
(86) PCT No.: PCT/EP2023/074890

§ 371 Date: Feb. 20, 2025

(30) Foreign Application Priority Data
Sep. 14, 2022 (IT) 102022000018756

(51) Int. Cl. A61M 1/00; A61F 13/00; A61F 13/05; A61M 25/00

(52) U.S. Cl. CPC A61M 1/912; A61F 13/05; A61F 2013/00536; A61M 2025/0059

(57) ABSTRACT

A flexible connector which can be coupled to a device for treating and removing a fluid from a wound using a negative pressure system is disclosed. The connector includes a deformable tubular cover having a first end connectable to a wound plaster and an opposite second end. The flexible connector can be coupled to a negative pressure source so that the fluid flows from the first end to the second end inside the tubular cover when the negative pressure is applied. An anti-collapse element placed inside the tubular cover prevents contact between the internal walls of the tubular cover when the negative pressure is applied and allows the passage of the fluid. The anti-collapse element includes a joining portion having a first extreme inside the tubular cover and a second extreme outside the tubular cover connectable to the negative pressure source.

### TECHNICAL FIELD

The present invention relates to a flexible connector which can be coupled to a device for treating a wound and removing a fluid from said wound by means of the use of a negative n pressure system, for example for treating wounds resulting from metabolic diseases such as diabetes or for treating any surgical incision. Furthermore, the present invention relates to a medical kit for treating a wound and removing a fluid from said wound by means of the use of a negative pressure system.

### STATE OF THE ART

The inflammation process which occurs during wound healing is characterized by excessive blood flow to the damaged tissue. Platelets tend to aggregate to form fibroblasts and new tissue while white blood cells and red blood cells represent the so-called exudate. If the wound is exposed to microorganisms or other external pathogens, an infection of the wound could develop, thus causing a worsening of the patient's condition and inevitably a lengthening of the healing times. Thus, the elimination of these pathogens from the damaged tissue accelerates and improves the wound healing process.

The use of negative pressure devices to treat wounds of various kinds has spread considerably over the last thirty years in combination with the use of electric vacuum generators. Negative pressure wound therapy has been shown to be extremely effective in treating acute, chronic or exudative wounds such as ulcers (pressure, diabetic or venous), surgical incisions and traumatic amputations.

The application of reduced or negative pressure creates a suction to remove excessive exudate from the wound, reducing the risk of maceration and infection. Furthermore, negative pressure stimulates the flow of fresh and oxygenated blood into the wound region as well as the formation of granulation tissue.

The negative pressure can be generated by devices of various kinds (with or without the use of electric pumps) which are connected, by means of a system of more or less rigid tubes, to a plaster applied directly to the wound to be treated. The tube system can comprise a flexible connector (known as soft port) in the end portion which is applied directly to the plaster.

Usually, negative pressure kits employ a generic tube of a generic plastic with a generic rigid fitting. This type of connection is very uncomfortable for the patient, creating pressure points or tension zones on the plaster. These pressure or tension zones are not comfortable for the patient. Such a problem has generated the need to create soft and more comfortable fluid connections. The softer and more flexible the connection, the more comfortable the dressing is for the patient.

More advanced kits use soft connectors to reduce the pressure zones and reduce stresses on the plaster.

Usually, the connectors in the negative pressure therapy which connect a traditional tube to a plaster consist of three layers. A first plastic layer, a second layer also in plastic and a third layer, or spacer layer, for example made of textile material (three-dimensional fabric or tubular fabric), positioned between the two plastic layers. The two plastic layers are generally bonded along the entirety of their perimeter so as to form a sort of pocket-like structure in which the spacer layer is inserted. Such a structure naturally has good flexibility along a longitudinal axis but is rigidly limited in the plane defined by the various layers. Therefore, the known flexible connectors are very wide and flexible in a single direction. It should be noted that these types of connectors are mainly adapted to suck and flow fluids of an aeriform nature and are absolutely not suitable for the passage of fluids containing a liquid substance. In fact, in the traditional systems, the exudate is generally absorbed by the plaster, i.e., by at least one absorbent layer of the plaster, so that the flexible connector essentially serves to suck air. In order to prevent the passage of liquid material beyond the flexible connector towards the device generating negative pressure, a hydrophobic filter can be placed between the flexible connection and the plaster. Having a flexible connector which is not capable of flowing liquids causes all the liquid material to concentrate in the absorbent layers present in the plaster and/or inside the connector itself, causing a swelling of these layers with a consequent decrease in the effectiveness of the sucking force with the passage of time during the treatment. Furthermore, a good adhesion of the plaster to the wound can be compromised due to the deformation of the plaster itself.

It is an object of the present invention to overcome in part or in whole the above-mentioned drawbacks of the known systems and to provide a flexible connector for negative pressure devices which is effective, safe, economical and at the same time easy to use. Furthermore, it is an object of the present invention to provide a flexible connector and a medical kit, comprising said connector and a plaster, which are capable of flowing a fluid which also contains liquid substances.

### DISCLOSURE OF THE INVENTION

A flexible connector and a medical I kit comprising said connector according to the independent claims are disclosed herein. Embodiments of the connector and kit are disclosed in the corresponding dependent claims.

In an aspect of the invention, the flexible connector can be coupled to a device for treating a wound and removing a fluid from said wound by means of the use of a negative pressure system. Said device can be a generic mechanical apparatus comprising for example a piston slidable inside a housing for generating the negative pressure. The device can however alternatively be an electrical vacuum generator. For example, the device can create a pressure of about −160 mmBar.

The connector comprises a deformable tubular cover having a first end which can be connected to a plaster to be applied to said wound and a second end opposite the first end. The connector is configured to be coupled to a negative pressure source such that the fluid flows from the first end to the second end inside the tubular cover when the negative pressure is applied, and an anti-collapse element positioned inside the tubular cover to prevent contact between the internal walls of the tubular cover when the negative pressure is applied and to allow the passage of fluid both in the absence and presence of the negative pressure.

Furthermore, the anti-collapse element comprises a joining portion having a first extreme inside the tubular cover and a second extreme outside the tubular cover connectable to the negative pressure source, in particular by means of a connection tube, wherein said joining portion is fixed to the tubular cover at a contact region, in particular at the first extreme, of said joining portion.

Since the anti-collapse element comprises a joining portion positioned between the inside and the outside of the tubular cover and is connected with the negative pressure source positioned outside the tubular cover, and since the joining portion is only fixed to the tubular cover at the contact region, the anti-collapse element is freer in movements and the connector can be easily deformed in all directions, i.e., along both the longitudinal axis and along the transverse plane. Moreover, the presence of so configured anti-collapse element avoids throttling points when a negative pressure is applied and when the tubular cover is crushed or folded. In addition, such a flexible connector is configured to allow the flow of any type of fluid, both aeriform and liquid in nature. Therefore, this flexible connector can advantageously be coupled to a negative pressure device capable of sucking liquids.

It should be noted that the flexible connector essentially consists of only two elements, namely the tubular cover and the anti-collapse element. In fact, the anti-collapse element is configured to connect directly to the negative pressure source by means of the joining portion which is an integral part of the anti-collapse element. In other words, the anti-collapse element extends from the inside to the outside of the tubular cover. Consequently, the flexible connector disclosed herein has a limited number of components (only two components) resulting in a reduction in production costs and a structural simplification of the connector itself.

In a second aspect of the invention, the medical kit is used for treating a wound and removing a fluid from said wound by means of the use of a negative pressure system. The medical kit comprises the flexible connector according to the preceding aspect and a plaster applicable to a wound and connected to the flexible connector at the first end of the deformable tubular cover of said flexible connector, wherein in particular the flexible connector is connected to the plaster by means of a connection cover to increase the adhesion between the flexible connector and the plaster and to protect the plaster in the event of traction of the flexible connector.

This kit is practical and functional. In fact, applying the plaster to the wound to be treated and connecting the flexible connector to a negative pressure source, it is possible to effectively remove the excessive exudate from the wound. Since the kit is configured in particular to also suck liquid substances, the plaster will not deform due to the absorption of the exudate and the adherence to the wound will remain optimal. Furthermore, the particular configuration of the flexible connector results in a smooth flow of the exudate throughout the treatment period.

Through the use of this connector and this kit, it is possible to reduce the risk of infections and the risk of complications arising. Thereby, wound healing is accelerated.

These and other aspects of the present invention will become more apparent by reading the following description of some preferred embodiments disclosed below.

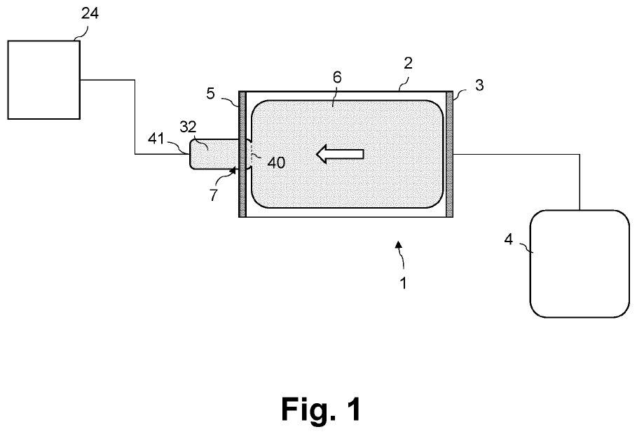

FIG. 1 shows a schematic depiction of the flexible connector connected to a plaster and a negative pressure source according to an example.

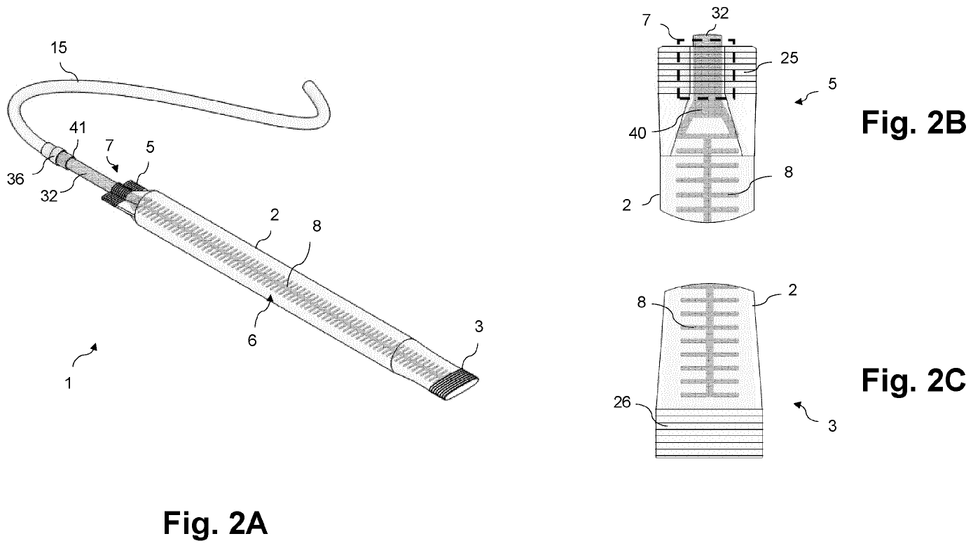

FIG. 2a-c show a schematic perspective depiction of the flexible connector according to an example as well as the details of the first and second end.

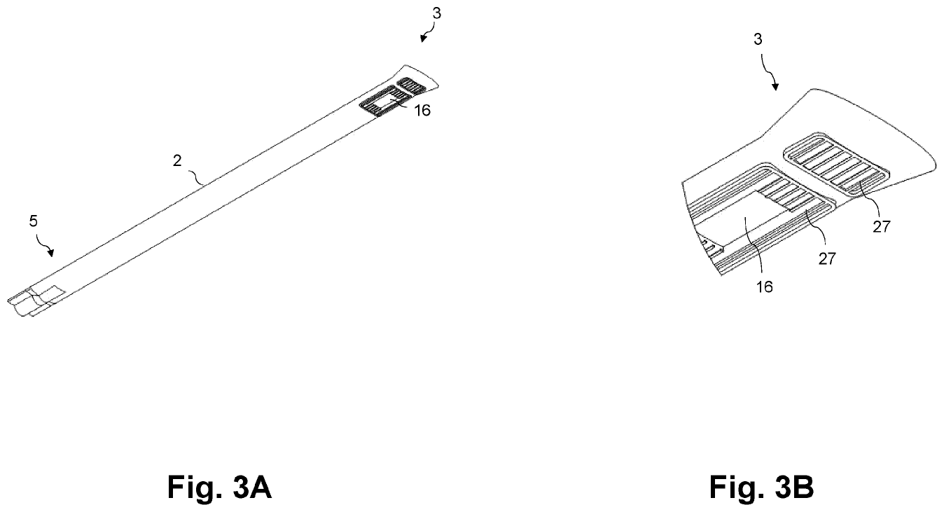

FIG. 3a-b show schematic perspective depiction and a detail of the first end viewed from below of the flexible connector according to an example.

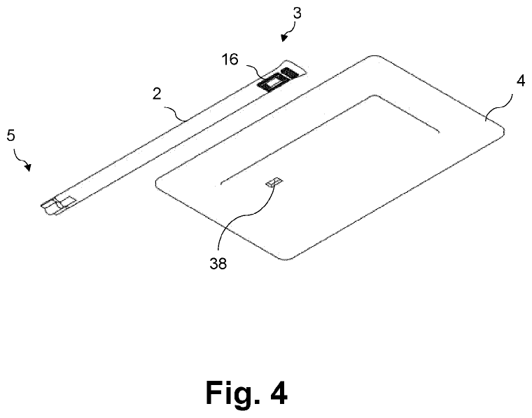

FIG. 4 shows a schematic perspective depiction of the flexible connector and the plaster according to an example.

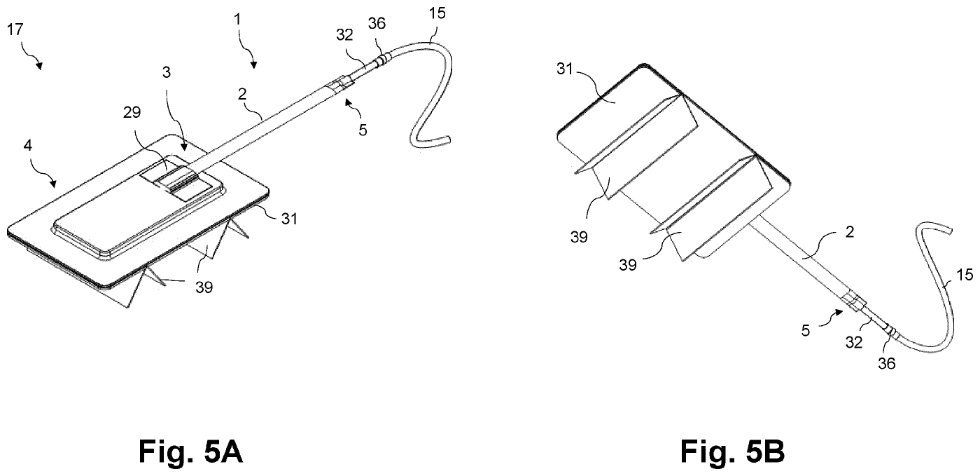

FIG. 5a-b show a schematic depiction of a medical kit according to an example seen from above and seen from below.

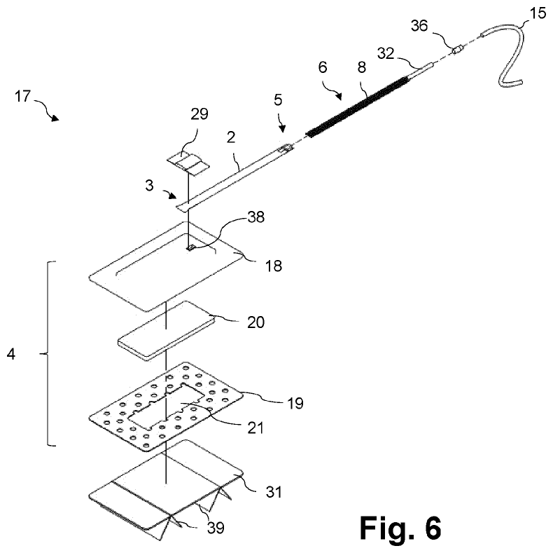

FIG. 6 shows a schematic depiction of the kit of FIG. 5 with exploded view.

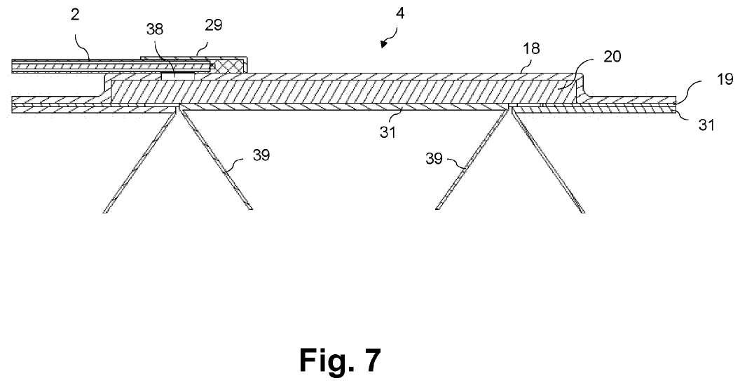

FIG. 7 shows a cross-section of the kit of FIG. 5 according to an example.

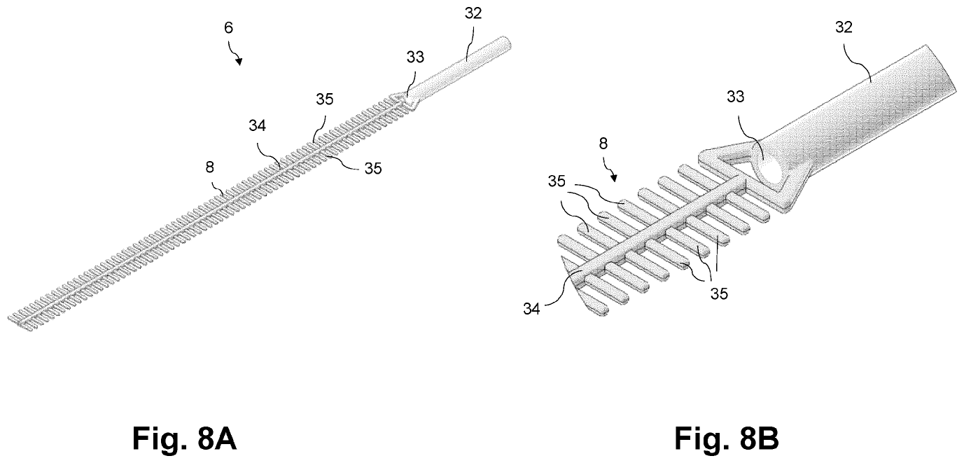

FIG. 8a-b show a schematic depiction of the anti-collapse element according to an example.

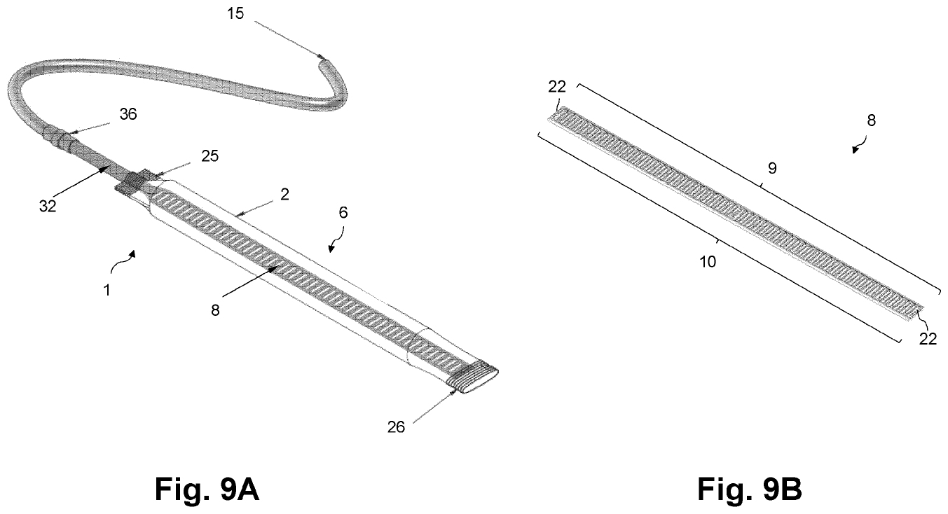

FIG. 9a-b show a schematic depiction of the flexible connector and the anti-collapse element according to another example.

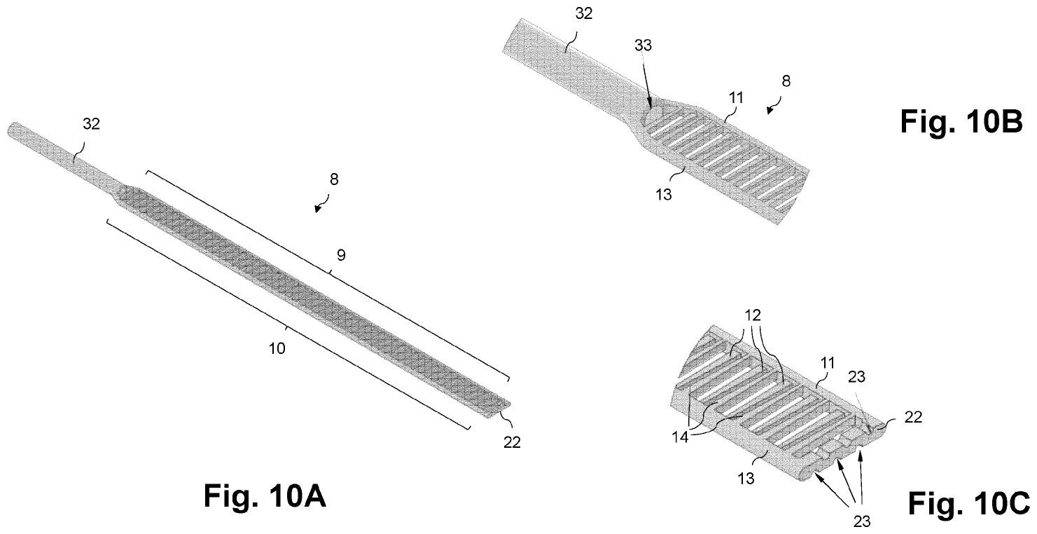

FIG. 10a-c show a schematic depiction of the anti-collapse element according to an example.

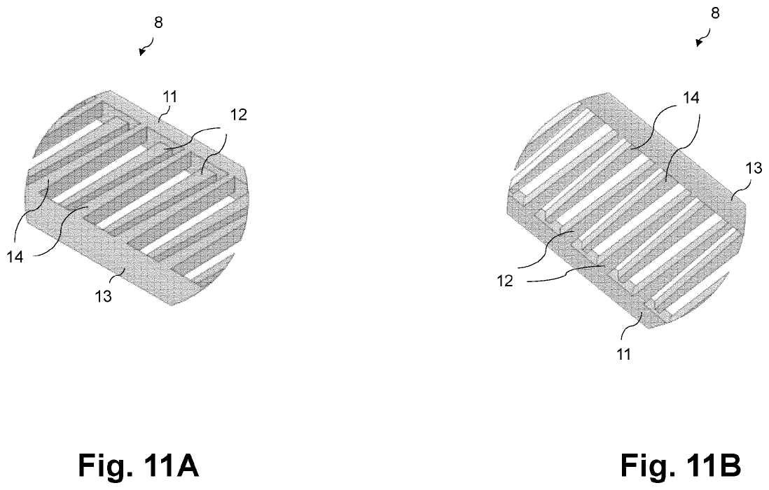

FIG. 11a-b show in a schematic depiction a detail of the anti-collapse element according to an example.

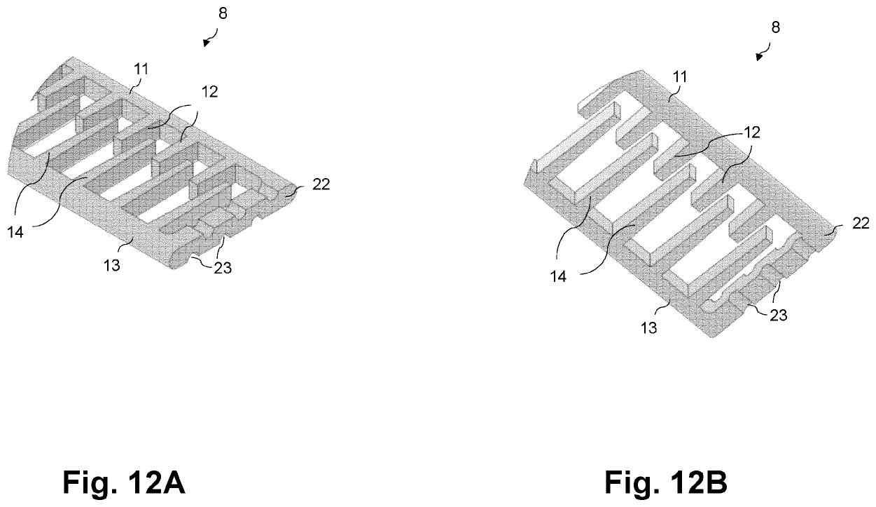

FIG. 12a-b show in a schematic depiction a detail of the anti-collapse element according to a further example.

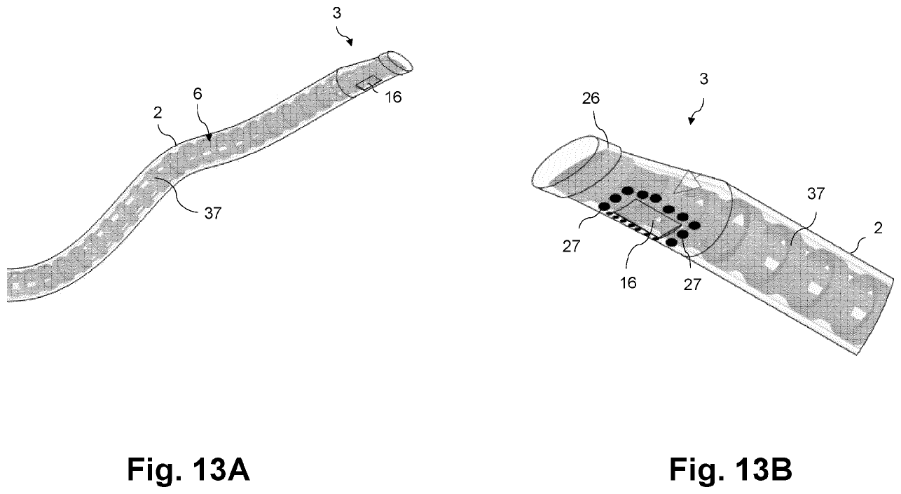

FIG. 13a-b show a schematic depiction of the flexible connector and the anti-collapse element according to a further example.

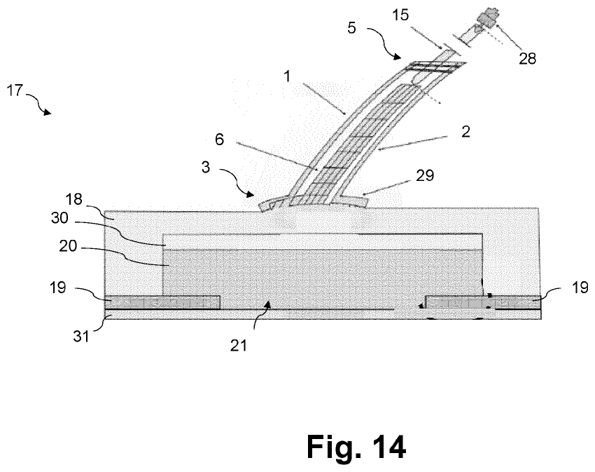

FIG. 14 shows a schematic depiction of a kit according to an example.

FIG. 1 shows a schematic drawing of the flexible connector 1 which can be coupled on one side to a negative pressure source 24 and on the other to a plaster 4 to be applied on the wound of a patient. The flexible connector 1 comprises a deformable tubular cover 2 with a first end 3 which can be connected to the plaster 4 and a second end 5 which is opposite the first end 3. The flexible connector 1 is configured to be coupled to the negative pressure source 24, for example to a device capable of generating a negative pressure. Thereby, the fluid can flow inside the tubular cover 2 from the first end 3 towards the second end 5, as shown by the arrow in the figure.

In order to prevent contact between the internal walls of the tubular cover 2 when the negative pressure is applied and to allow the passage of the fluid both in the absence and in the presence of negative pressure, the flexible connector 1 comprises an anti-collapse element 6 placed inside the tubular cover 2. The anti-collapse element 6 comprises a joining portion 32 for connecting to the negative pressure source 24. The joining portion 32 comprises a first extreme 40 located inside the tubular cover 2 and a second extreme 41 located outside the tubular cover 2 connected to the negative pressure source 24. The joining portion 32 is fixed to the tubular covering 2 at a contact region 7 of said joining portion (32).

It should be noted that the coupling between the second end 5 of the tubular cover 2 and the negative pressure source 24 can occur indirectly by means of, for example, the joining portion 32 of the anti-collapse element 6.

The contact region 7 can be located at any point along the extension of the joining portion 32. For example, as shown in the figure, the contact region 7 is located near or at the first extreme 40.

In n example, the anti-collapse element 6 is fixed to the tubular cover 2 only at the second end 5 of said tubular cover 2 by means of the joining portion 32. This means that the anti-collapse element 6, although it may be in contact with any area of the tubular cover 2, is not actually fixed thereto at any point. The only point of contact between the anti-collapse element 6 and the tubular cover 2 occurs by means of the joining portion 32 at the contact region 7. This contact point represents a fixing point between the anti-collapse element 6 and the tubular cover 2, for example by means of welding. This ensures greater flexibility of the connector 1, as the presence of the anti-collapse element inside the tubular cover 2 thus configured does not limit the movements thereof (twisting, folding, rotation, etc.). Alternatively, the anti-collapse element 6 can additionally be fixed to the tubular cover 2 at the first end 3 as well.

Advantageously, the connector 1 consists solely of the deformable tubular cover 2 and the anti-collapse element 6. With respect to the flexible connectors present in the literature, the number of components used is reduced to two, thus making the connector 1 according to the present disclosure simpler and cheaper to manufacture. Furthermore, the limited number of components reduces the risk of damage. In addition, reliability is improved with respect to the traditional connectors which have a large region of welds along the perimeter of the connection (about 40-50 cm) which, if not performed correctly, can compromise the effective pneumatic seal of the connector. The connector 1 according to the present description limits the number of welds to the initial and final part only (about 2 cm each).

According to an example, the tubular cover 2 is made in one piece, having a circular section when no negative pressure is applied. In other words, unlike the flexible connectors known in the literature, when at rest, the tubular cover 2 is circular in shape rather than flat. In particular, the tubular cover is made by means of extrusion. The tubular cover 2 is extruded in one piece with this shape to essentially facilitate production and assembly. Naturally, once the negative pressure is applied, the tubular cover 2 deforms, assuming a crushed (i.e., oval) section. However, the presence of the anti-collapse element 6 avoids a contact between the internal walls of the tubular cover 2. It should be noted that by using a linear extrusion, the tubular cover 2 can be made of a highly flexible material, for example plastic.

Since the tubular cover 2 is made in one piece and the only coupling points with the anti-collapse element 6 are at its ends (for example only at the second end 5 or at the first and second end 3, 5), assembly is facilitated and performance is improved. In fact, the absence of welding/gluing along the lateral margins of the tubular cover 2 increases the useful section for the passage of fluid and the flexibility of the connector 1. In other words, the entire section of the tubular cover 2 is available for the passage of the fluid, thus also allowing a reduction in the thickness and total width of the flexible connector 1, specifically of the component for the fluid connection. Given the great flexibility, in the event of mechanical stress, e.g., crushing due to negative pressure, the cover 2 will tend to flatten locally, in particular according to different directions at different points in the event of external pressure. This is certainly more convenient and advantageous than a flexible connector which is initially flat which instead has large flexibility limitations in some directions.

In an example, the anti-collapse element 6 is made of a non-absorbent material. For example, the anti-collapse element 6 can be made of a plastic polymeric material. Thereby, the fluid, also containing liquid substances, can flow inside the tubular cover 2 without being blocked, as it is absorbed by the anti-collapse element 6. Additionally or alternatively, the tubular cover 2 is internally devoid of any absorbent material. This means that the fluid present inside the tubular cover 2, even containing liquid substances, can flow freely from one end to the other without being intercepted by any absorbent element.

FIG. 2a shows an example of the flexible connector 1. From the figure it can be seen that the tubular cover 2 of the connector 1 is one piece and has a roughly circular section which flattens at the ends. The first end W of the tubular cover 2 is illustrated as a free end in the figure but is must be understood as an end to be coupled to the plaster 4. The second end 5 is instead connected to a connection tube 15 which can be used for a coupling with a negative pressure source 24. Specifically, the connection tube 15 is connected by means of the connector 36 to the joining portion 32 of the anti-collapse element 6, in particular to the second extreme 41 of said portion 32.

In an example, the anti-collapse element 6 comprises a flat semi-rigid structure 8, in particular made of polymeric material. Its purpose is to avoid the collapse of the walls of the tubular cover 2 and the blocking of the fluid communication. The fixing to the second end 5 of the tubular cover 2 is shown in FIG. 2b. At this end 5, the tubular cover 2 comprises a closure 25 wherein the tubular cover 2 and the joining portion 32 are fixed together, for example by means of welding. Specifically, the fixing between the tubular cover 2 (for example in polyurethane) and the joining portion 32 occurs at the contact region 7 (dashed area in FIG. 2b) by means of radio-frequency welding, electro-welding or gluing. FIG. 2b also shows that the joining portion 32 forms a single element together with the flat semi-rigid structure 8. In fact, at the first extreme 40, the joining portion 32 which has a roughly tubular shape widens and flattens to become the flat semi-rigid structure 8 of the anti-collapse element 6. The joining portion 32 thus represents an end of the anti-collapse element 6 which exits outside the tubular cover 2 and which ensures the contact/fixing with said tubular cover 2 at the contact region 7. Furthermore, the joining portion 32 ensures the connection with the connection tube 15 and therefore the passage of the fluid. FIG. 2c shows a detail of the tubular cover 2 and of the anti-collapse element 6 at the first end 3. In particular, the tubular cover 2 comprises a closure 26 made for example by means of welding. The anti-collapse element 6, or rather the end part of the flat semi-rigid structure 8, can be inserted inside the closure 26. This serves to avoid that, in the event of mechanical stress on the longitudinal axis, the tubular cover 2 deforms while the anti-collapse element 6 moves therein. It should be noted that in this case the end part of the anti-collapse element is simply inserted inside the fold formed by the closure 26 to keep the anti-collapse element in position, without however there being a fixing between the tubular cover 2 and the anti-collapse element 6 at the first end 3. Alternatively, the end part of the anti-collapse element 6 can be fixed for example by means of welding to the tubular cover 2 at the closure 26. In an example, the first end 3 of the tubular cover 2 is closed, in particular by means of gluing, heat-sealing, radiofrequency, and/or electro-welding.

FIG. 3a shows the tubular cover 2 seen from below. It is also evident from this figure how the tubular cover 2 is made in one piece, or is not formed by two or more overlapping layers joined (welded) for example along the longitudinal edges. It should be noted that the first end 3 of the tubular cover 2 comprises a hole 16 for the passage of the fluid inside the connector 1. This is shown in detail in FIG. 3b. The hole 16 is therefore positioned on a lower side of the tubular cover 2 so that said tubular cover 2 can be easily applied on a plaster 4. FIGS. 3a and 3b show a hole 16 of rectangular shape. It is however evident that the shape, as well as the dimensions, of the hole 16 can vary according to structural requirements. In order to attach the tubular cover directly to the plaster 4, or to a material interposed with the plaster 4, there is an adhesive layer 27 at the hole 16. To increase the adhesion, the figure shows how the adhesive layer 27 is located around the perimeter of the hole 16 and along an adjacent strip. Naturally, the adhesive layer 27 can be distributed according to different configurations (for example only in some points along the perimeter of the hole 16 and/or distributed on more or less extensive surfaces near the hole 16). Furthermore, the adhesive layer 27 can be replaced with suitable alternative fixing means.

FIG. 4 shows the tubular cover 2 of the flexible connector 1 and a plaster 4 to which the connector 1 can be coupled. In particular, the plaster 4 comprises an inlet 38 on a region of its upper surface. The connection between the connector 1, i.e., the tubular cover 2, and the plaster 4 occurs by coupling the first end 3 of the tubular cover 2 to the inlet 38 of the plaster 4. Specifically, it occurs by coupling the hole 16 to the inlet 38. The adhesive layer 27 serves to fix the coupling between the tubular cover 2 and the plaster 4.

FIGS. 5-7 show a medical kit 17. The kit 17 represents a set of components ready for use and comprises at least one flexible connector 1 and a plaster 4 applicable on a wound and connected to the flexible connector 1 at the first end 3 of the deformable tubular cover 2 (as disclosed with reference to FIG. 4). It should be noted that these figures show the use of the connector 1 having an anti-collapse element 6 as disclosed in FIG. 2a. However, the kit 17 is not limited to this single configuration of the anti-collapse element 6, i.e., comprising the flat semi-rigid structure 8. In fact, flexible connectors 1 having different anti-collapse elements 6 can also be used inside the medical kit 17, as will be disclosed later. From FIG. 5a it should be noted that the flexible connector 1 is coupled to the plaster 4 at an end thereof. Specifically, the first end 3 of the tubular cover 2 is fixed to an upper region of the plaster 4, for example by means of the adhesive layer 27, as shown in FIG. 4. The other end of the connector 1 can be coupled to a connection tube 15 for the connection to a negative pressure source 24.

Specifically, the joining portion 32 can be fixed to the connection tube 15 by means of a connector 36 as already shown in FIG. 2a. The plaster 4 can comprise a support layer (carrier) 31 which is easily detachable and serves to maintain the plaster 4 in its flat form during storage. As shown in FIG. 5b, the carrier 31 advantageously comprises support tabs 39 which can serve to facilitate the assembly between plaster 4 and connector 1. The support tabs 39 are foldable and can be crushed under the plaster 4 if necessary.

FIG. 6 shows an exploded view of the medical kit 17 of FIG. 5. It should be noted how the flat semi-rigid structure 8 representing the anti-collapse element 6 can be easily inserted inside the tubular cover 2 thanks to the configuration of the latter, i.e., the creation as a single piece of circular section. To increase the adhesion between the flexible connector 1 and the plaster 4 and to protect the plaster 4 in the event of traction of the flexible connector 1, the flexible connector 1 can optionally be connected to the plaster 4 by means of a connection cover 29. This cover 29 or “connection patch” can have a square or rectangular shape, for example with a 3 cm side, applied directly to the upper side of the plaster 4.

According to an example, the plaster 4 consists of a first

layer 18 made of polymeric material, in particular polyurethane, a second layer 19 in contact with the first layer 18 and made of silicone adhesive material (for example “Acrysil 150 perforated” from Advanced Silicone Coating as a function of its excellent seal due to the trilaminate and locally perforated structure) to attach the plaster 4 to the epidermis of a patient, and a third layer 20 arranged between the first layer 18 and the second layer 19 to facilitate the passage of fluid, wherein the second layer 19 comprises a central opening 21 to allow the contact of a portion of the third layer 20 with the patient's wound. The second layer 19 can be a perforated layer.

In Other Words, the Plaster 4 Is Essentially a Sandwich structure of three elements: a polyurethane layer (first layer) 18, a layer of perforated silicone glue (second layer) 19, and a three-dimensional spacer element (third layer 20). These layers are coupled to each other by means of lamination (e.g., cold) and with an acrylic adhesive 30. The acrylic adhesive 30 is generally present on the lower side of the first layer 18, as shown for example in FIG. 14. However, the adhesive 30 can also be present on the upper face of the second layer 19. The layer 19 of perforated silicone glue serves to have a delicate and durable adhesion on the skin and to allow the plaster 4 to be repositioned, in the event first incorrect application, as this adhesive prevents the first polyurethane layer 18 from curling and sticking to itself (electrostatic forces, gravity, mechanical deformation).

FIG. 7 shows a cross-section of the kit 17, in particular of the tubular cover 2 of the connector 1 coupled to the plaster 4 at the inlet 38 of the plaster 4. The layered structure of the plaster and the reduced total thickness can be appreciated from FIG. 7.

The advantages of using the spacer element (i.e., the third layer 20) inside the plaster 4 are at least the following:
the upper face in contact with the acrylic adhesive 30 and the first polyurethane layer 18 is configured so as to have an optimal adhesion;
the lower face is configured with a reduced surface tension to have a weak adhesion on the side of the wound and thus reduce the risk of damage during dressing change operations;
it is formed by a material which does not absorb liquids, for example silicone material, to improve the drainage of fluids from the wound to the suction device;
it is also very flexible, generally increasing the flexibility of the plaster 4 and thus the ability to accommodate the non-planarity of the human body;
a homogenization of the pressure over the entire area of the plaster 4 is determined (optimal pressure), as it is formed by a single element with continuous properties over the entire surface (a plaster wherein this element is formed by several layers of different materials would be less effective in this task, given the different properties of the aforesaid layers).

In an example, the flat semi-rigid structure 8 is a fishbone structure comprising a central longitudinal element 34 and two sets of teeth 35 extending orthogonally from two opposite sides of said central longitudinal element 34. This configuration is shown in FIGS. 8a and 8b. The figures show how the anti-collapse element 6 comprises the joining portion 32 integrated with the fishbone flat semi-rigid structure 8.

Using the fishbone structure, it is possible to use a smaller amount of material used in production (time and cost) with respect to other types of structures. The fishbone structure, having only a central channel, allows an easy insertion inside the tubular cover 2 (made of polyurethane), since the “spines” of the fishbone, i.e., the teeth 35, fold when the element is inserted. Moreover, having only a central core, this central longitudinal element 34 is the one which manages to ensure the greatest flexibility of the connector 1. Lastly, the fishbone structure facilitates the passage of fluid (air and/or liquid) from the plaster to the suction device, as it maximizes the detachment of the walls of the tubular cover 2.

As already seen above, the joining portion 32 is made in one piece with the anti-collapse element 6, in particular with the flat semi-rigid structure 8, in particular by means of injection molding.

In an example, the joining portion 32 is provided with a through hole 33 which extends along the entire tubular portion 32. The detail of the through hole 33 is visible in FIG. 8b. Thereby, in the passage from the first end 3 to the second end 5 inside the tubular cover 2 when the negative pressure is applied, the fluid initially flows surrounding the anti-collapse element 6 and then flows inside said anti-collapse element 6 at the joining portion 32 through the hole 33.

An extreme (first extreme 40) of the tubular portion 32 is fixable to the tubular cover 2 at the second end 5 of said s tubular cover 2, in particular by means of electro-welding, chemical welding, radiofrequency welding, and/or gluing.

FIG. 9a shows a flexible connector 1 comprising an anti-collapse element 6 with a flat semi-rigid structure 8 which extends inside the tubular cover 2 and which is different from the flat semi-rigid structure 8 of FIG. 8a.

As already disclosed, the closure at the second end 25 allows the fixing between the joining portion 32 and the tubular cover 2, for example by means of welding. The closure at the first end 26 can occur by means of welding the tubular cover 2 and is such that the end of the anti-collapse element 6 (flat semi-rigid structure 8) ends where the welding of the tubular cover 2 begins.

As in the previous configuration, the tubular portion 32 is made in one piece or in one component, with the anti-collapse element 6, in particular with the flat semi-rigid structure 8, in particular by means of injection molding. This greatly facilitates assembly, avoiding having to immobilize the anti-collapse element 6 inside the tubular cover 2 and improves general performance. Since an extreme (first extreme 40) of the joining portion 32 is fixable to the tubular cover 2 at the second end 5 (closure at the second end 25) and the joining portion 32 is fixed to the anti-collapse element 6 (is made in one piece therewith), the anti-collapse element 6 is indirectly connected with the tubular cover 2 only at the second end 5 by means of the joining portion 32. Alternatively, the anti-collapse element 6 can be additionally fixed to the tubular cover 2 also at the first end 3.

The flat semi-rigid structure 8 according to an example is shown in detail in FIGS. 9b, 10a-c and 11a-b.

It should be noted that the flat semi-rigid structure 8 of FIG. 9b does not comprise an integrated joining portion 32, as in the embodiment of FIG. 10a. The structure of FIG. 9b is only an alternative embodiment, wherein the joining portion 32 can be connected (i.e., removably fixed) to one of the two ends of the flat semi-rigid structure 8 to make a connection with the negative pressure source 24 as disclosed above.

Although this alternative exists, the preferred solution for the flexible connector 1 is to have an anti-collapse element 6 having a joining portion 32 integrated in a single component with the flat semi-rigid structure 8.

In an example, the flat semi-rigid structure 8 is a double comb structure comprising a first comb component 9 having a first longitudinal element 11 from which a plurality of first teeth 12 extends and a second comb component 10 having a second longitudinal element 13 from which a plurality of second teeth 14 extends, wherein the first comb component 9 is connected to the second comb component 10 at the ends of the first longitudinal element 11 and the second longitudinal element 13, and wherein the first comb component 9 is coupled to the second comb element 10 such that at least one portion of a tooth of the plurality of first teeth 12 is arranged in a space between two consecutive teeth of the plurality of second teeth 14.

With a double comb structure it is possible to obtain advantages related to industrial production, as this structure is the most easily and quickly moldable by means of polymer injection. Furthermore, the fact of not having “protrusions” on the outer sides but being smooth facilitates the insertion inside the tube 2.

FIGS. 9b and 10a show in particular how the flat semi-rigid structure 8 is formed by two comb components 9, 10, wherein the teeth of the one are inserted between the spaces of the teeth of the other. FIG. 9b specifically shows a structure wherein the two comb components 9, 10 are symmetrically connected to each other at the two ends by means of two contact cross members 22. FIG. 10a instead shows a configuration wherein one of the two ends is integral with the joining portion 32 provided with a through hole 33. Therefore, there is only one contact cross member 22. FIGS. 10b and 10c respectively depict a detail of the structure of FIG. 10a at the two ends.

For example, the flat semi-rigid structure 8 is formed by a double comb coupled to form a ladder-like element, wherein the pegs are alternately connected to one or the other upright. This allows the fluid connection despite the possible partial collapse of the tubular cover 2. The use of a particularly elastic and deformable polymer allows good comfort and excellent deformability. This structure facilitates assembly operations, as it does not need a guide for the insertion outside the tubular polyurethane cover 2, as instead occurs for the systems known in the literature. The flat semi-rigid structure 8 can be produced by means of injection molding of polymers, allowing to have a component with very low cost and easy and fast reproducibility. The coupling between the tubular polyurethane cover 2, the anti-collapse element 6 with flat semi-rigid structure 8 and thus the joining portion 32 can occur in the same manner as disclosed above, i.e., by means of electro-welding, radiofrequency or gluing. A connection tube 15 can be directly fixed (glued) to the joining portion 32 by means of a suitable connector 36.

FIG. 10b shows in particular the presence of the through hole 33 which crosses the entire joining portion 32 for its length and connects the connection tube 15 (when connected to the flexible connector 1 as shown in FIG. 9a) to the flat semi-rigid structure 8 (for example with a double comb) of the anti-collapse element 6.

According to an example, as anticipated, the first comb component 9 is connected to the second comb component 10 by means of at least one contact cross member 22, wherein said contact cross member 22 comprises at least one recess 23 to facilitate the passage of the fluid. FIG. 10c shows the presence of six recesses 23 arranged in an offset manner on the two sides of the contact cross member 22. The number of recesses 23 varies according to structural needs.

The fluid, also containing liquid substances, can thus effectively flow through the recesses 23, along the teeth of the flat semi-rigid structure 8, and through the through hole 33, to then outflow in the connection tube 15.

From the figures it should be noted that the plurality of first teeth 12 and the plurality of second teeth 14 have a tapered shape, i.e., a shape which gradually narrows, for example with a triangular section. This facilitates the fluid flow. Furthermore, both the plurality of first teeth 12 and the plurality of second teeth 14 join the first longitudinal element 11 to the second longitudinal element 13. This joining can occur in such a way that the tip of the teeth of the plurality of first teeth 12 is an integral part of the second longitudinal element 13 or is simply in contact therewith. Similarly, the joining can occur in such a way that the tip of the teeth of the plurality of second teeth 14 is an integral part of the first longitudinal element 11 or is simply in contact therewith.

Alternatively, the length of each of the teeth of the plurality of first teeth 12 and/or the plurality of second teeth 14 can be less than the distance between the first longitudinal element 11 and the second longitudinal element 13 such that the tip of the teeth of the plurality of first teeth 12 is not in contact with the first longitudinal element 11 and/or the tip of the teeth of the plurality of second teeth 14 is not in contact with the second longitudinal element 13. This improves i.e., increases the flexibility of the anti-collapse element 6.

In an example, the plurality of first teeth 12 lies on a plane offset from the plane on which the plurality of second teeth 14 lies. This is illustrated in FIGS. 11a and 11b which show a detail of the flat semi-rigid structure 8 as seen from above and as seen from below. It should be noted that the plurality of first teeth 12 lies on a lower plane with respect to the plurality of second teeth 14. This offset improves the flow of fluid during treatment, as the offset planes improve the spacing of the internal walls of the flexible tube 2.

FIGS. 12a and 12b illustrate a different configuration of the double-comb flat semi-rigid structure 8. In an example, the plurality of first teeth 12 has a different length than the length of the plurality of second teeth 14. Thereby the flexibility of the anti-collapse element 6 is increased and the passage of the fluid during the treatment is improved. In fact, with this configuration the separation of the internal walls of the tube 2 is increased. Furthermore, it allows an easy passage of material inside the fluid (solid particles such as clusters of cells and coagulated blood).

It should be noted that the various configurations of the flat semi-rigid structure 8 shown in FIGS. 8-12 can be alternative or combinable with each other. One or more embodiments combining some or all configurations are conceivable. For example, the flat semi-rigid structure 8 can comprise at least one double-comb portion and one fishbone portion, wherein the double-comb portion can comprise a plurality of teeth having the same length or different lengths as disclosed above.

FIGS. 13a and 13b show a further configuration of the anti-collapse element 6. According to an example, the anti-collapse element 6 is deformable and comprises a tubular structure 37, in particular a woven element with circular knit. This tubular structure 37 can be a “textile tube”, a 3D spacer, or a simple woven element with circular knit. In particular, analogously to the configuration of FIGS. 3a and 3b, the first end 3 of the tubular cover 2 comprises a hole 16 for the passage of the fluid inside the connector 1. In order to attach the tubular cover directly to the plaster 4, or to a material interposed with the plaster 4, there is an adhesive layer 27 at the hole 16. To increase the adhesion, the figure shows how the adhesive layer 27 is located in a series of points distributed around the perimeter of the hole 16.

FIG. 14 shows the medical kit 17 in a schematic depiction. The medical kit 17 essentially comprises the flexible connector 1 and a plaster 4 coupled to the connector 1. FIG. 12 generally shows an anti-collapse element 6 with a reticular structure connected to the connection tube 15 at the second end 5 of the tubular cover 2. However, as mentioned above, the flexible connector 1 can comprise only some or all of the features disclosed above. For example, the flexible connector 1 can comprise an anti-collapse element 6 having a tubular structure 37 or a flat semi-rigid structure 8 (or both). Furthermore, the flat semi-rigid structure 8 can have a double comb shape or a fishbone shape (or both).

The plaster 4 comprises a first layer 18 made of polymeric material, in particular polyurethane, a second layer 19 in contact with the first layer 18 and made of silicone adhesive material for attaching the plaster 4 to the epidermis of a patient, and a third layer 20 arranged between the first layer 18 and the second layer 19 to facilitate the passage of fluid. To ensure the contact of a portion of the third layer 20 with the patient's wound, the second layer 19 comprises a central opening 21. The first layer 18 can be attached to the third layer 20 by means of an acrylic adhesive.

The first layer 18 represents a barrier for bacteria and viruses of various kinds. Furthermore, it is resistant to water. The thickness of the first layer 18 (without the acrylic adhesive 30 of 20 g/m²) is comprised between 20 μm and 30 μm. The breathability of this layer is MVTR 800/600.

The third layer 20 is made of non-absorbent material so as to allow the passage of the fluid when the negative pressure is applied. In particular, this layer 20 has a very low absorption rate, close to zero and a thickness of about 3 mm.

The plaster can lastly comprise a support layer (carrier) 31 which is easily detachable and serves to maintain the plaster in its flat form during storage and to avoid exposure of the third layer 20 prior to use.

The kit 17 can also be provided with a connection tube 15 and a check valve 28 at an end of the connection tube 15.

A person skilled in the art can perform several and further modifications and variants to the connector 1 and to the kit 17 disclosed above, in order to satisfy further and contingent needs, all said modifications and variants however included within the scope of protection of the present invention as defined by the appended claims.

1. A flexible connector which can be coupled to a device for treating a wound and removing a fluid from said wound by means of the use of a negative pressure system, the connector comprising:
a deformable tubular cover having a first end which can be connected to a plaster to be applied to said wound and a second end opposite the first end wherein the flexible connector is configured to be coupled to a negative pressure source such that the fluid flows from the first end to the second end inside the tubular cover when the negative pressure is applied, and
an anti-collapse element placed inside the tubular cover to prevent contact between the internal walls of the tubular cover when the negative pressure is applied and to allow the passage of the fluid both in the absence and in the presence of negative pressure,
wherein
the anti-collapse element comprises a joining portion having a first extreme inside the tubular cover and a second extreme outside the tubular cover connectable to the negative pressure source in particular by means of a connection tube wherein said joining portion is fixed to the tubular cover at a contact region in particular at the first extreme of said joining portion.

2. The connector according to claim 1, wherein
a. the anti-collapse element is fixed to the tubular cover only at the second end of said tubular cover by means of the joining portion and/or
b. the connector consists solely of the deformable tubular cover and the anti-collapse element.

3. The connector according to claim 1, wherein the tubular cover is made in one piece having a circular section when the negative pressure is not applied, and wherein the tubular cover is made by means of extrusion.

4. The connector according to claim 1, wherein
a. the anti-collapse element is made of a non-absorbent material, and/or
b. the tubular cover is internally devoid of any absorbent material.

5. The connector according to claim 1, wherein the anti-collapse element is deformable and comprises a flat semi-rigid structure and comprises polymeric material.

6. The connector according to claim 5, wherein the flat semi-rigid structure is a fishbone structure comprising a central longitudinal element and two sets of teeth extending orthogonally from two opposite sides of said central longitudinal element.

7. The connector according to claim 5, wherein the flat semi-rigid structure is a double comb structure comprising a first comb component having a first longitudinal element from which a plurality of first teeth extends and a second comb component having a second longitudinal element from which a plurality of second teeth extends, wherein the first comb component is connected to the second comb component at the ends of the first longitudinal element and the second longitudinal element and wherein the first comb component is coupled to the second comb element such that a portion of a tooth of the plurality of first teeth is arranged in a space between two consecutive teeth of the plurality of second teeth

8. The connector according to claim 7, wherein the first comb component is connected to the second comb component by a contact cross member wherein said contact cross member comprises a recess to facilitate the passage of the fluid.

9. The connector according to claim 7, wherein
a. the plurality of first teeth lies on a plane offset from the plane on which the plurality of second teeth lies, and/or
b. the plurality of first teeth has a different length with respect to the length of the plurality of second teeth

10. The connector according to claim 1, wherein
a. the joining portion is made in one piece with the anti-collapse element having a flat semi-rigid structure by means of injection molding; and/or
b. the joining portion is fixable to the tubular cover by means of electro-welding, chemical welding, radiofrequency welding, and/or gluing.

11. The connector according to claim 1, wherein the joining portion of the anti-collapse element is provided with a through hole which extends along the entire joining portion and wherein, in the passage from the first end to the second end inside the tubular cover when the negative pressure is applied, the fluid initially flows surrounding the anti-collapse element and then flows inside said anti-collapse element at the joining portion through the through hole.

12. The connector according to claim 1, wherein the first end of the tubular cover is closed, in particular by gluing, heat-welding and/or electro-welding, and comprises a hole for the passage of fluid inside the connector

13. A medical kit for treating a wound and removing a fluid from said wound by means of the use of a negative pressure system, the kit comprising:
the flexible connector according to one claim 1; and
a plaster applicable to a wound and connected to the flexible connector at the first end of the deformable tubular cover of said flexible connector wherein the flexible connector is connected to the plaster by means of a connection cover to increase the adhesion between the flexible connector and the plaster and to protect the plaster in the event of traction of the flexible connector

14. The kit according to claim 13, wherein the plaster consists of a first layer made of polymeric material, a second layer in contact with the first layer and made of silicone adhesive material to attach the plaster to the epidermis of a patient, and a third layer arranged between the first layer and the second layer to facilitate the passage of fluid, wherein the second layer comprises a central opening to allow contact of a portion of the third layer with a patient's wound.

15. The kit according to claim 13, wherein the third layer is made of non-absorbent material so as to allow the passage of the fluid when the negative pressure is applied.
